(19) United States
(12) Patent Application Publication
(10) Pub. No.: US 2006/0293259 A1
(43) Pub. Date: Dec. 28, 2006

(54) Compositions and methods of use of derivatized flavanols

(75) Inventors: Catherine L. Kwik-Uribe, Stroudsburg, PA (US); Harold H. Schmitz, Bethesda, MD (US); Mark A. Kelm, East Stroudsburg, PA (US); John F. Hammerstone JR., Rockville, MD (US)

Correspondence Address: NADA JAIN, P.C., 560 White Plains Road, Suite 460, Tarrytown, NY, 10591, US

(73) Assignee: Mars, Incorporated, McLean, VA (US)

(21) Appl. No.: 11/476,500

(22) Filed: Jun. 28, 2006

Related U.S. Application Data
(60) Provisional application No. 60/694,629, filed on Jun. 28, 2005.
(60) Provisional application No. 60/754,007, filed on Dec. 23, 2005.

(51) Int. Cl. A61K 31/7048; A61K 31/353

(52) U.S. Cl. 514/27; 514/456

(57) ABSTRACT

The invention relates to compositions containing derivatized flavanols such as methylated flavanols, and methods of use thereof for prophylactic or therapeutic treatment of a human or a veterinary animal for example as anti-platelet agents.

This application claims the benefit, under 35 USC Section 119, of the U.S. Provisional Application Ser. No. 60/694,629 filed Jun. 28, 2005, and Provisional Application Ser. No. 60/754,007 filed Dec. 23, 2005, the disclosures of both are hereby incorporated herein by reference.

### FIELD OF THE INVENTION

The invention relates to compositions containing derivatized flavanols, (e.g. alkylated, alkenylated, and alkynylated flavanols) such as methylated flavanols, and methods of use thereof for prophylactic or therapeutic treatment of a human or a veterinary animal for example as anti-platelet agents.

### BACKGROUND OF THE INVENTION

Some polyphenols, such as flavanols and procyanidins, have been shown to have a beneficial effect on the inhibition of platelet aggregation and hence on treatment of a variety of health conditions that have platelet aggregation as one of the underlying risk factors. For example, blood platelets play a major role in coronary artery disease. Platelets are found at the site of atherosclerotic lesions. When activated, they secrete potent mitogenic factors such as platelet derived growth factor, transforming growth factor-β and epidermal growth factor, which lead to smooth muscle proliferation and progression of atherosclerotic lesions. Additionally, enhanced platelet reactivity and spontaneous platelet aggregates are crucially involved in thrombus formation, which is largely responsible for the pathogenesis of acute myocardial infarction, unstable angina and percutaneous coronary intervention. Therapy with antiplatelet agents (such as aspirin) significantly decrease the incidence of primary and secondary coronary events (Schafer, A. I. “Antiplatelet Therapy”, A. J. Med. 101:199-209, 1996).

Platelet function depends on the interactions of membrane glycoproteins, such as GPIIb/IIIa, which act as receptors for adhesive proteins on the platelet surface. Agonists of GPIIb/IIIa facilitate the conformational change necessary for the receptors to become receptive to the ligands which bind simultaneously to two separate platelets, thereby cross-linking and aggregating the platelets. Antagonists of the GPIIb/IIIa receptor prevent the activation of the receptor, thereby preventing platelet activation and/or aggregation. Pharmocologic intervention directed against the GPIIb/IIIa receptor is therefore being pioneered in the treatment of ischemic heart disease. Several GPIIb/IIIa antogonists have been used in clinical trials in recent years, and have been shown to have considerable benefit in various treatment regimes (Vorchheimer et al, JAMA, 281:15:1407-1413, 1999).

Given that the diseases mentioned above are life threatening, there remains a need in the art for anti-platelet agents. Applicants have now discovered that derivatized flavanols, such as alkylated, alkenylated, and alkynylated flavanols, may be used for anti-platelet therapy.

### SUMMARY OF THE INVENTION

The invention relates to derivatized flavanols, (e.g. alkylated, alkenylated, and alkynylated flavanols), a composition comprising an effective amount of a derivatized flavanol and methods of use thereof for antiplatelet therapy.

In one aspect, the invention relates to a composition, such as a pharmaceutical, a food, a food additive, or a dietary supplement comprising an effective amount of a derivatized flavanol (e.g. alkylated, alkenylated, and alkynylated flavanols). Also within the scope of the invention are packaged products containing the above-mentioned compositions and a label and/or instructions for use to treat or prevent platelet aggregation and related conditions.

In another aspect, the invention relates to methods of use of derivatized flavanols (e.g. alkylated, alkenylated, and alkynylated flavanols), to treat or prevent platelet aggregation and related conditions.

### BRIEF DESCRIPTION OF THE DRAWINGS

FIGS. 1a-1 to 1c represents the results of platelet aggregation experiments with 3′-O-methyl catechin, 4′-O-methyl catechin and 4′-O-methyl epicatechin.

FIG. 2a-g represents the results of platelet aggregation and leukocyte activation experiments with 3′-O-methyl catechin, 4′-O-methyl catechin and 4′-O-methyl epicatechin.

### DETAILED DESCRIPTION

All patents, patent applications and references cited in this application are hereby incorporated herein by reference. In case of any inconsistency, the present disclosure governs.

The invention relates to a derivatized flavanol, (e.g. alkylated, alkenylated, and alkynylated flavanols), compositions comprising an effective amount of the derivatized flavanol, or a pharmaceutically acceptable salt or derivative thereof, and methods of use thereof for anti-platelet therapy.

The compound of the present invention is a derivatized flavanol or a pharmaceutically acceptable salt or derivative thereof (including oxidation products and glucuronidated products) having the following formula: 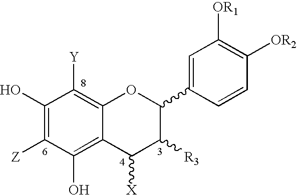
wherein
(i) R₁ or R₂ or both are selected from the group of: C₁ to C₄ alkyl (C₁, C₂, C₃, or C₄ alkyl, i.e. methyl, ethyl, propyl or butyl), C₃ to C₄ alkenyl, and C₃ to C₄ alkynyl; with the proviso that when R₁ or R₂ or both are C₃ to C₄ alkenyl, or C₃ to C₄ alkynyl, the unsaturated carbons are separated by at least one carbon from the oxygen atom;
(ii) R₃ is -(α)-OH, -(β)-OH, -(α)-O-sugar, -(β)-O-sugar, -(α)-O-gallate, or -(β)-O-gallate;
(iii) each X, Y or Z is a hydrogen or a sugar; and
(iv) when R₁ or R₂ is not C₁ to C₄ alkyl, C₃ to C₄ alkenyl, or C₃ to C₄ alkynyl, it is a hydrogen.
   For example, R₁ or R₂ or both in the above formula are C₁ to C₄ alkyl, e.g. —CH₃. In other embodiments, R₁ or R₂ or both in the above formula are C₃ to C₄ alkenyl. In yet other embodiments, R₁ or R₂ or both in the above formula are C₃ to C₄ alkynyl.

In some embodiments, the compound is a derivatized flavanol or a pharmaceutically acceptable salt or derivative thereof (including oxidation products and glucuronidated products) having the following formula: 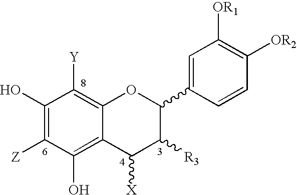
wherein
(i) R₁ or R₂ or both are selected from the group of: C₁ to C₄ alkyl (C₁, C₂, C₃, or C₄ alkyl, i.e. methyl, ethyl, propyl or butyl), C₃ to C₄ alkenyl, and C₃ to C₄ alkynyl; with the proviso that when R₁ or R₂ or both are C₃ to C₄ alkenyl, or C₃ to C₄ alkynyl, the unsaturated carbons are separated by at least one carbon from the oxygen atom;
(ii) R₃ is -(α)-OH, -(β)-OH, -(α)-O-sugar, -(β)-O-sugar, -(α)-O-gallate, or -(β)-O-gallate;
(iii) X, Y and Z are hydrogen; and
(iv) when R₁ or R₂ is not C₁ to C₄ alkyl, C₃ to C₄ alkenyl, or C₃ to C₄ alkynyl, it is a hydrogen.
   For example, R₁ or R₂ or both in the above formula are C₁ to C₄ alkyl, e.g. —CH₃. In other embodiments, R₁ or R₂ or both in the above formula are C₃ to C₄ alkenyl. In yet other embodiments, R₁ or R₂ or both in the above formula are C₃ to C₄ alkynyl.

In yet other embodiments, the compound is a derivatized flavanol or a pharmaceutically acceptable salt or derivative thereof (including oxidation products and glucuronidated products) having the following formula: 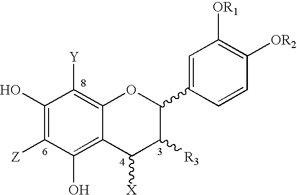
wherein
(i) R₁ or R₂ or both are selected from the group of: C₁ to C₄ alkyl (C₁, C₂, C₃, or C₄ alkyl, i.e. methyl, ethyl, propyl or butyl), C₃ to C₄ alkenyl, and C₃ to C₄ alkynyl; with the proviso that when R₁ or R₂ or both are C₃ to C₄ alkenyl, or C₃ to C₄ alkynyl, the unsaturated carbons are separated by at least one carbon from the oxygen atom;
(ii) R₃ is -(α)-OH, or -(β)-OH;
(iii) X, Y and Z are hydrogen; and
(iv) when R₁ or R₂ is not C₁ to C₄ alkyl, C₃ to C₄ alkenyl, or C₃ to C₄ alkynyl, it is a hydrogen.
   For example, R₁ or R₂ or both in the above formula are C₁ to C₄ alkyl, e.g. —CH₃. In other embodiments, R₁ or R₂ or both in the above formula are C₃ to C₄ alkenyl. In yet other embodiments, R₁ or R₂ or both in the above formula are C₃ to C₄ alkynyl.

In the above structural formulas a C₃ alkyl (i.e., propyl) group may be n-propyl or iso-propyl. A C₄ alkyl (i.e., butyl) group may be n-butyl, sec-butyl or tert-butyl.

The sugar is preferably a monosaccharide or di-saccharide. The sugar can be selected from the group consisting of glucose, galactose, rhamnose, xylose, and arabinose. The sugar may optionally be substituted with a phenolic moiety at any position, for instance, via an ester bond. The phenolic moiety is selected from the group consisting of caffeic, cinnamic, coumaric, ferulic, gallic, hydroxybenzoic and sinapic acids.

Examples of derivatives include esters, oxidation products and glucuronidated products. Oxidation products may be prepared as disclosed in U.S. Pat. No. 5,554,645, the relevant portions of which are incorporated herein by reference. Esters, for example esters with gallic acid, may be prepared using known esterification reactions, and for example, methods as described in U.S. Pat. No. 6,420,572, the disclosure of which is hereby incorporated herein by reference. Glucuronidated products may be prepared as described in Yu et al, “A novel and effective procedure for the preparation of glucuronides.” Organic Letters, 2(16) (2000) 2539-41. Glucuronidation may take place at the 7, 5 and/or 3′ position(s). Examples of glucuronidated products include 4′-O-methyl-epicatechin-O-β-D-glucuronide (e.g. 4′-O-methyl-epicatechin-7-O-β-D-glucuronide), 3′-O-methyl-epicatechin-O-β-D-glucuronide (e.g. 3′-O-methyl-epicatechin-5/7-O-β-D-glucuronides), and epicatechin-O-β-D-glucuronide (e.g. epicatechin-7-O-β-D-glucuronide).

Also within the scope of the invention are C₁ to C₄ ether alcohol derivatives of flavanols as shown in some of the examples below.

Examples of the compounds of the invention are as follows:
(i) 3′-O-methyl-catechin or 3′-O-methyl-epicatechin, 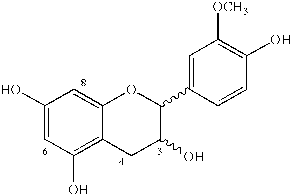
(ii) 4′-O-methyl-catechin or 4′-O-methyl-epicatechin, 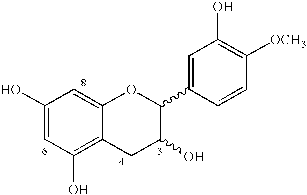
(iii) 3′-O-4′-O-dimethyl-catechin or 3′-O-4′-O-diethyl-epicatechin,

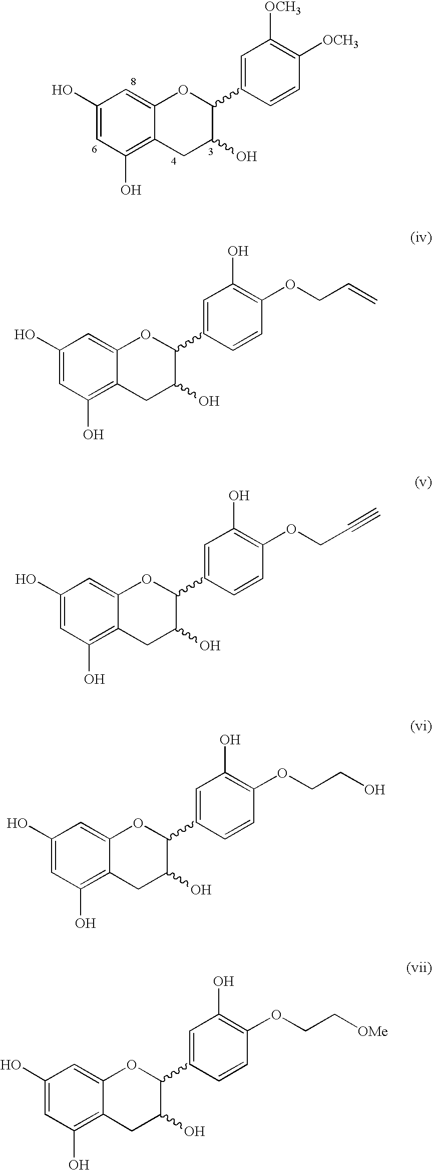

Further examples of derivatized flavanols are 3′-O-methyl-(+)catechin or 3′-O-methyl-(−)epicatechin, 4′-O-methyl-(+)catechin or 4′-O-methyl-(−)epicatechin, and 3′-O—, 4′-O-dimethyl-(+)catechin or 3′-O—, 4′-O-dimethyl-(−)epicatechin.

The compounds can be prepared synthetically and purified using the methods described in Example 1 and/or as described in the art (see e.g., Olive, et. al., J. Chem Soc., Perkins Trans. 1:821-830, 2002), relevant portions of which are hereby incorporated herein by reference, or may be isolated from natural sources using known sources (e.g. cinnamon) and methods (see, e.g., Morimoto, et. al., Chem. Pharm. Bull. 33(6) 2281-2286, 1985), relevant portions of which are hereby incorporated herein by reference.

Compositions comprising an effective amount of any of the compounds described herein are also within the scope of the invention.

### Methods of Use

Any compound and/or composition described in the application may be used to practice the methods described herein.

The invention relates to a method of anti-platelet therapy comprising administering to a subject in need thereof an effective amount of any of the compounds described above, wherein the subject is a human or veterinary animal. For example, a subject in need of anti-platelet therapy suffers from, or is at risk of suffering from, thrombosis; plaque rupture; atherosclerosis; cardiovascular disease (CVD); coronary artery disease (CAD) (including myocardial ischemia, myocardial infarction, stable and unstable angina, acute occlusion or restenosis), diabetes (type I and type II) (e.g. vascular complications of diabetes), cognitive dysfunction or disorder and/or vascular circulation disorders (including those of the brain), heart attack, cerebrovascular disease (including stroke, initial and/or recurrent transient ischemic attack, or ischemic complications e.g. complications after coronary angioplasty or percutaneous coronary intervention), post-operative injury (e.g. postoperative ischemia and/or thrombosis or inflammation), congestive heart failure, kidney failure, renal failure; peripheral artery disease; non-rheumatic atrial fibrillation; and acute coronary syndrome.

As used herein, “treatment” means improving an existing medical condition, for example, by slowing down the disease progression, prolonging survival, reducing the risk of death, and/or inducing a measurable decrease in platelet activation and/or aggregation.

The term “preventing” means reducing the risks associated with developing a disease, including reducing the onset of the disease. For example, subjects having a family medical history of conditions recited herein may be suitable for prophylactic treatment. Generally, any subject having at least one of the cardiovascular disease risk factors (as recognized by the American Heart Association) may be treated as described herein.

The effective amount for use in the above methods may be determined by a person of skill in the art using the guidance provided herein and general knowledge in the art. For example, the effective amount may be such as to achieve a physiologically relevant concentration in the body (e.g. blood) of a mammal. Such a physiologically relevant concentration may be at least about 10 nanomolar (nM), preferably at least about 20 nM, or at least about 100 nM, and more preferably at least about 500 nM. In one embodiment, at least about one micromole in the blood of the mammal, such as a human, is achieved. The compounds of the formula, as defined herein, may be administered at from about 50 mg/day to about 1000 mg/day, preferably from about 100-150 mg/day to about 900 mg/day, and most preferably from about 300 mg/day to about 500 mg/day. However, amounts higher than stated above may be used.

The compounds of the invention may be administered acutely, or treatment/preventive administration may be continued as a regimen, i.e., for an effective period of time, e.g., daily, monthly, bimonthly, biannually, annually, or in some other regimen, as determined by the skilled medical practitioner for such time as is necessary to achieve therapeutic or prophylactic effects. The administration may be continued for at least a period of time required to exhibit therapeutic/prophylactic effects. Preferably, the composition is administered daily, most preferably two or three times a day, for example, morning and evening to maintain the levels of the effective compounds in the body of the mammal. To obtain the most beneficial results, the composition may be administered for at least about 30, or at least about 60 days. These regiments may be repeated periodically.

Any of the above methods may be practiced using the compounds of the invention and at least one additional therapeutic agent. Such therapeutic agents may include therapies that are known to inhibit platelet aggregation, as well as any other therapeutics, especially those that treat conditions resulting from or affected by platelet aggregation.

### Compositions and Formulations

The compositions of the invention may be administered as a pharmaceutical, food, food additive or a dietary supplement.

As used herein a “food” is a material containing protein, carbohydrate and/or fat, which is used in the body of an organism to sustain growth, repair and vital processes and to furnish energy. Foods may also contain supplementary substances such as minerals, vitamins and condiments. See Merriam-Webster's Collegiate Dictionary, 10th Edition, 1993. The term food includes a beverage adapted for human or animal consumption. A “food additive” is as defined by the FDA in 21 C.F.R. 170.3(e)(1) and includes direct and indirect additives. A “pharmaceutical” is a medicinal drug. See Merriam-Webster's Collegiate Dictionary, 10th Edition, 1993. A pharmaceutical may also be referred to as a medicament. A “dietary supplement” is a product (other than tobacco) that is intended to supplement the diet that bears or contains the one or more of the following dietary ingredients: a vitamin, a mineral, an herb or other botanical, an amino acid, a dietary substance for use by man to supplement the diet by increasing the total daily intake, or a concentrate, metabolite, constituent, extract or combination of these ingredients.

Pharmaceuticals containing the inventive compounds, optionally in combination with another therapeutic agent, may be administered in a variety of ways such as orally, sublingually, bucally, nasally, rectally, intravenously, parenterally and topically. A person of skill in the art will be able to determine a suitable mode of administration to maximize the delivery of derivatized flavanols, optionally in combination with another therapeutic agent. Thus, dosage forms adapted for each type of administration are within the scope of the invention and include solid, liquid and semi-solid dosage forms, such as tablets, capsules, gelatin capsules (gelcaps), bulk or unit dose powders or granules, emulsions, suspensions, pastes, creams, gels, foams, jellies or injection dosage forms. Sustained-release dosage forms are also within the scope of the invention. Suitable pharmaceutically acceptable carriers, diluents, or excipients are generally known in the art and can be determined readily by a person skilled in the art. The tablet, for example, may comprise an effective amount of the derivatized flavanol containing composition and optionally a carrier, such as sorbitol, lactose, cellulose, or dicalcium phosphate.

The foods comprising a derivatized flavanol and optionally another therapeutic or beneficial-to-health agent (e.g. flavanols, A-type or B-type procyanidins) may be adapted for human or veterinary use, and include pet foods. The food may be other than a confectionery, for example, a beverage. A confectionery such as a standard of identity (SOI) and non-SOI chocolate, such as milk, sweet and semi-sweet chocolate including dark chocolate, low fat chocolate, a candy (e.g. a candy bar) which may be a chocolate covered candy comprising the composition of the invention is also within the scope of the invention. Other food examples include a baked product (e.g. brownie, baked snack, cookie, biscuit) a condiment, a granola bar, a toffee chew, a meal replacement bar, a spread, a syrup, a powder beverage mix, a cocoa or a chocolate flavored beverage, a pudding, a rice cake, a rice mix, a savory sauce and candy bars, such as granola bars, containing nuts, for example, peanuts, walnuts, almonds, and hazelnuts. If desired, the foods may be chocolate or cocoa flavored.

The dietary supplement containing derivatized flavanol, and optionally another therapeutic or beneficial-to-health agent, may be prepared using methods known in the art and may comprise, for example, dicalcium phosphate, magnesium stearate, calcium nitrate, vitamins, and minerals.

Further within the scope of the invention is an article of manufacture such as a packaged product comprising the composition of the invention (e.g. a food, a dietary supplement, a pharmaceutical) and a label indicating the presence of, or an enhanced content of the inventive compounds, or directing use of the composition for anti-platelet therapy, e.g. methods of treatment and/or prophylaxis of thrombosis; plaque rupture; atherosclerosis; cardiovascular disease (CVD); coronary artery disease (CAD) (including myocardial ischemia, myocardial infarction, stable and unstable angina, acute occlusion or restenosis), diabetes (type I and type II) (e.g. vascular complications of diabetes), cognitive dysfunction or disorder and/or vascular circulation disorders (including those of the brain), heart attack, cerebrovascular disease (including stroke, initial and/or recurrent transient ischemic attack, or ischemic complications e.g. complications after coronary angioplasty or percutaneous coronary intervention), post-operative injury, congestive heart failure, kidney failure, renal failure; peripheral artery disease; non-rheumatic atrial fibrillation; and acute coronary syndrome. The packaged product may contain the composition and the instructions for use. The label and/or instructions for use may refer to any of the methods of use described in this application. The invention also relates to methods of manufacturing the article of manufacture comprising any of the compositions described herein, packaging the composition to obtain an article of manufacture and instructing, directing or promoting the use of the composition/article of manufacture for the uses described herein. Such instructing, directing or promoting includes advertising.

Also within the scope of the invention is an article of manufacture (such as a packaged product or kit) adapted for use in combination therapy comprising at least one container and at least one derivatized flavanol, or a pharmaceutically acceptable salt or derivative thereof. The article of manufacture further comprises at least one additional vascular health protective agent (i.e., other than the derivatized flavanol, or a pharmaceutically acceptable salt or derivative thereof), which agent may be provided as a separate composition, in a separate container, or in admixture with the compound of the invention. Examples of other therapeutic anti-platelet therapy agents are COX inhibitors, such as aspirin and anticoagulants/blood thinning agents such as warfarin and heparin.

In certain embodiments, therapeutic agents optionally administered with derivatized flavanol may be flavanols, A-type or B-type procyanidins, for example cocoa flavanols and/or procyanidins which can be prepared as is known in the art (see, e.g. U.S. Pat. Nos. 5,554,645; 6,297,273; 6,420,572; 6,156,912; 6,476,241; 6,864,3776; 670,390; and 6,015,913).

The invention is further described in the following non-limiting examples.

### EXAMPLES

### Example 1

### Synthesis, Purification and Structural Identification of 3′ and 4′-O-Alkylated (−) Epicatechin

### Materials and Methods

Chemicals

HPLC grade methanol, acetonitrile, acetone, isopropanol and acetic acid were purchased from Fischer Scientific (Boston, Mass.). (−) Epicatechin, iodoethane, iodomethane and potassium carbonate were purchased from Aldrich-Sigma Chemical Co. (St. Louis, Mo.). Deuterated NMR solvents (d₄-MeOH, d₆-acetone, d₃-ACN) were purchased from Cambridge Isotope Laboratories (Andover, Mass.) and Aldrich-Sigma Chemical Co.

Synthesis

Anhydrous K₂CO₃ (6.9 g) was magnetically stirred into acetone (250 mL). Epicatechin (2.5 g) was then added and stirred (5-10 min). While stirring, CH₃I or CH₃CH₂I (10 mL) was added slowly. Reaction was carried out at ambient temp in a sealed flask. Reaction was monitored by HPLC-MS in negative ion mode every 2-4 hours until the ratio of epicatechin ([M−1]⁻; m/z 289), 3′-O-Me-epicatechin ([M−1]⁻; m/z 303), and 4′-O-Me-epicatechin ([M−1]⁻; m/z 303) were approximately 1:1:1. The reaction of CH₃CH₂I with epicatechin was monitored in a similar fashion in accordance with expected molecular ions. The crude products were worked up by vacuum filtration of the reaction mixture through a Büchner funnel with a Whatman #4 filter to remove K₂CO₃ solids. Acetone was removed by rotary evaporation under reduced pressure at 40° C. Solids were dissolved in isopropanol then filtered as before to remove any residual K₂CO₃. Solvents were removed by rotary evaporation under reduced pressure at 40° C. to afford a pale brown crusty residue. The synthesis described above was adapted from previously published work (Donovan, L. R., Luthiria, D. L., Stremple, P., Waterhouse, A. L. “Analysis of (+) catechin, (−) epicatechin and their 3′-and 4′-O-methylated analogs, A comparison of sensitive methods.” Journal of Chromatography B, 726 (1999) 277-283.

3′,4′-O-dimethyl epicatechin may also be synthesized by the above described method.

Purification

The purification system consisted of two Agilent 1100 Preparative Pumps (Agilent Technologies, Wilmington, Del.), Agilent 1100 keypad controller, Rheodyne injection valve fitted with a 5 mL loop (Rhonert Park, Calif.), HP1050 UV detector (Hewlett Packard, Palo Alto, Calif.), Luna 10μ Prep C18 (2) 250×50 mm column (Phenomenex, Torrance, Calif.), and a Kipp and Zonen flatbed recorder (Bohemia, N.Y.). Eluents were monitored at 280 nm. Peaks corresponding to compounds of interest were collected, rotary evaporated under reduced pressure at 40° C. to remove organic solvents, then freeze-dried to remove water. Other purification details of epicatechin metabolites are described below.

The crude product mixture of 3′- and 4′-O-Me-epicatechin was purified by gradient elution of B (ACN) into A (0.1% HOAc in H2O) at 30 mL/min. The gradient was 0-30 min; 28.0-30.0% B, 30-30.01 min; 30.0-50.0% B, 30.01-35 min; 50-100%, 35-40 min; 100-28%, 40-45 min; 28% B. The purification of 3′- and 4′-O-ethyl-epicatechin was facilitated by isocratic elution (71:29, 0.1% HOAc in H2O:ACN) of crude reaction mixture at a flow rate of 30 mL/min.

Structural Determination

Analyses of isolated compounds were performed using an Agilent 1100 HPLC coupled to an Agilent 1100 MSD/LC Trap equipped with an API-ES chamber. Compounds were subjected to reverse phase (RP) gradient chromatography over ODS Hypersil 5 microns 100×4.6 mm (Thermo Electron Corp.) at 20 C. The binary solvent system consisted of A (0.1% HOAc in H2O, v/v) and B (0.1% HOAc in MeOH, v/v). The gradient was 0-20 min; 15-25% B, 20-30 min; 25-50% B, 30-35 min 50-100% B with a flow rate of 1 mL/min. Conditions for the mass spectral analysis in negative ion mode included a capillary voltage of 4000 V, a nebulizing pressure of 40 psi, a drying gas flow of 12 L/min and a temperature of 350° C. Data was collected scanning over a mass range of m/z 120-700 at 3 s/cycle using Agilent ChemStation and Brucker Quant Analysis software. Nuclear magnetic resonance (NMR) spectra were obtained on a Brucker 500 MHz instrument (Brucker, Karlsruhe, Germany). ¹HNMR and ¹³CNMR spectra were recorded in d4-MeOH or d₆-acetone.

Results

Structural Elucidation

Structural elucidation of 3′-O and 4′-O-alkylated products was based upon theoretical order of elution, mass spectral data and ¹HNMR and ¹³CNMR experiments. Reverse phase order of elution using conditions described above of O-alkylated compounds (min) was: 3′-O-methyl epicatechin (19.8), 4′-O-methyl epicatechin (24.7), 3′-O-ethyl epicatechin (25.8), and 4′-O-ethyl epicatechin (28.8). Epicatechin eluted at 11.7 min. Order of elution was dictated by position and length of alkyl group. Compounds O-alkylated at the 3′ position were eluted sooner due to polarity of the 4′-OH. Increasing chain length of O-alkylated compounds enhanced retention by allowing greater partitioning into the stationary phase. The negative API-ES spectra of 3′ and 4′-O-methyl epicatechin both showed a deprotonated molecular ion (m/z 303) in agreement with mono-O-methyl epicatechin. Moreover, the retro-Diels Alder fragment ions (m/z 137) supported O-methylation only on the B-ring. The ¹HNMR chemical shifts and coupling constants of 3′ and 4′-O-methyl epicatechin were similar to those of epicatechin but differences can be explained in terms of the presence and position of the electron withdrawing —OCH₃ substituent. The closer aromatic protons were to the —OCH₃ group the greater the electron withdrawing effect via the sigma system, the greater the deshielding and thus the larger the downfield shift relative to epicatechin. Chemical shifts for H-2′, 5′ and 6shifted slightly downfield due to deshielding effects of —OCH₃ group at C-3′. Further downfield shifting of H-5′ and 6′ was observed in the ¹HNMR spectrum of 4′-O-methyl-epicatechin. Intense singlets (δ3.84 and 3.84) integrating for three protons each in ¹HNMR spectra for the two mono-O-methyl epicatechins were diagnostic of protons on —OCH₃. A total of 16 peaks with 15 chemical shifts similar to epicatechin were present in both ¹³CNMR spectra of 3′ and 4′-O-methyl epicatechins. Chemical shifts at 656.4 and 56.5 in each of the spectra were typical of —OCH₃ carbons.

Differentiation between 3′-O and 4′-O-ethyl epicatechin was based on theoretical order of elution as described above. The negative API-ES spectra of 3′ and 4′-O-ethyl epicatechin both showed a deprotonated molecular ion (m/z 317). The retro-Diels Alder fragment ion (m/z 137) supported O-ethylation only on the B-ring. ¹HNMR chemical shifts and coupling constants of both O-ethylated compounds were similar to epicatechin. Quartets (δ4.02, 4.04) and triplets (61.31, 1.33) corresponding to —OCH₂— and —CH₃ portion of —OCH₂CH₃ moieties were present in the ¹HNMR spectra of 3′- and 4′-O-ethyl epicatechin. Downfield shifts for H-2′, 5′ and 6′ can be explained in terms of presence and position of the electron withdrawing group —OCH₂CH₃ similar to —OCH₃ substituted analogs. H-8 and H-6 meta couplings were not observed for 3′-O-ethyl epicatechin. ¹³CNMR experiments also substantiated presence of —OCH₂CH₃ by the presence of peaks corresponding to —OCH₂— (δ64.9, 65,0) and —OCH₃ (δ14.9, 15.0) in the spectra of 3′- and 4′-O-ethyl epicatechin.

### Example 2

### Effects of Methylated Flavanols on Platelets in Whole Blood

Platelet aggregation was measured using a platelet counting technique, and formation of platelet/monocyte conjugates (P/M) and platelet/neutrophil conjugates (P/N) by flow cytometry. In later experiments the activation state of platelets associated with leukocytes (CD62P) was also measured and also the activation state of the leukocytes themselves (CD11b).

### Materials and Methods

Flavanols tested for inhibitory effect on platelet aggregation were: (+) catechin, [CAT+], (−) catechin [CAT−], (−) epicatechin [EP−], 3′OMe catechin [3mCAT], 4′OMe catechin [4mCAT] and 4′OMe epicatechin [4mEPCAT]. All agents were dissolved in ethanol, with full dissolution in some cases being achieved by sonication. Once in solution, further dilution with saline was possible. Hirudin, (Revasc™) was obtained from Novartis (Basel, Switzerland) and was stored as a 5 mg/ml solution in saline in a glass vial at −20° C. Collagen (Nycomed) was from Axis Shield Diagnostics (Dundee, UK). Concentrations were prepared from the stock solution (1 mg/ml) using the isotonic glucose buffer supplied by the manufacturer. Aspirin (acetyl salicylic acid-ASA), adenosine diphosphate (ADP), platelet activating factor (PAF), arachidonic acid (AA) and epinephrine were from Sigma. Fixing solution consisted of 140 mM NaCl containing 0.16% w/v formaldehyde, 4.6 mM Na₂EDTA, 4.5 mM Na₂HPO4 and 1.6 mM KH₂PO4, pH 7.4.

Blood samples were studied using the Multi-Sample Agitator (MSA) produced by the Medical Engineering Unit (University of Nottingham). The MSA is used to maintain blood samples at 37° C. and to agitate small samples of blood at a stir speed of 1,000 rpm as required.

Flow cytometry was carried out using commercially available fluorescent labelled antibodies on a Facscan (Becton Dickinson, UK) equipped with a 5 W laser operating at 15 mW power and a wavelength of 488 nM or an LSRII flow cytometer (Becton Dickinson, UK) equipped with an additional red Trigon laser operating at a wavelength of 633 nM.

Blood Collection

Blood was obtained from healthy volunteers, who denied taking any aspirin or non-steroidal anti-inflammatory drugs (NSAID) in the previous 10 days. This blood was dispensed into graduated polystyrene tubes that contained hirudin (final concentration 50 μg/ml) and a small volume of the flavanol under investigation or ethanol as control. The final concentration of ethanol in the blood was always 0.3%. In some experiments, aspirin (ASA) or saline as control was also included in the tube. The tubes were then capped and inverted three times to ensure adequate mixing then placed in the MSA at 37° C. for 30 min before the experiments were performed, during which time the blood was left undisturbed. A further sample of blood was taken into a commercially prepared vacutainer tube that contained K₂EDTA as anticoagulant.

Platelet Aggregation

Following a 30 min pre-incubation period, aliquots of blood (480 μl) were dispensed into small polystyrene tubes each containing a stir bar and stirred for 2 min in the MSA. After 2 min a solution (20 μl) of agonist or vehicle control were added to the tubes. These were then stirred in the MSA for up to 10 min at which time the platelet aggregates were fixed by mixing a small sub sample with fixative solution in a 1:2 ratio (v/v). The platelet count in the fixed samples was determined using the UltraFlo-100 Whole Blood Platelet Counter. Platelet aggregation was calculated as the percentage loss of single platelets with reference to the platelet count of the EDTA sample.

Platelet-Leukocyte Conjugate Formation

Platelet-leukocyte conjugate formation was measured in the same stirred samples used to measure platelet aggregation. Sub samples were taken 4 min or 10 min following the addition of agonist and transferred into the appropriate antibody or antibody mixture. These were then incubated in the dark at room temperature for not less than 20 min. Following red cell lysis and a washing procedure the cell suspensions were applied to either the FACScan or the LSRII flow cytometer. Leukocytes were identified by logical gating from dot plots of forward scatter (cell size) and side scatter (cell granularity) profiles acquired with linear amplification. Monocytes were identified by their forward scatter-side scatter profile and CD14 (PE) positivity, while neutrophils were identified in the same way but were negative for CD14 expression. The “pan” leukocyte marker, CD45 (PerCP) was also used to identify the leukocyte population. Fluorescence parameters were acquired with logarithmic amplification. Platelet monocyte (P/M) and platelet neutrophil (P/N) conjugates were quantified as median CD42a (FITC) fluorescence of the monocyte (P/M mf) or neutrophil population (P/N mf). Leukocyte activation was measured by CD11b (AlexaFluor647) expression (CD11b-M for monocytes and CD11b-N for neutrophils). Platelet activation (P-selectin expression) was measured by CD62P (PE) positivity of the platelets associated with leukocytes as (CD62P-M on P/M and CD62P-N on P/N).

The FACScan was used to measure the fluorescent probes in experiments where three colors were used together, but the LSRII was needed in order to study four colors. The LSRII is a more sensitive machine and produces higher fluorescence values (mf) than the FACScan. Results obtained on the FACScan cannot be directly compared with the results obtained on the LSRII.

### Results

Comparison of the Effects of Flavanols on Aggregation, P/M and P/N

Blood was obtained from three different volunteers and the platelet aggregation and platelet/leukocyte conjugate formation was measured in response to collagen (0, 0.125, 0.25 and 0.5 μg/ml). In this experiment the highest collagen concentration used previously (1 μg/ml) was replaced with a lower concentration of collagen (0.125 μg/ml) to optimise the inhibition brought about by the different flavanols. In these experiments (+) catechin (Sigma) was used at 1 mM, aspirin (100 μM) and the test flavanols at 0.3 mM with the exception of EP−(0.1 mM) and 4 mCAT (0.05 mM). Aggregation was measured at 4 and 10 min following agonist addition and platelet/leukocyte conjugate formation only at 10 min.

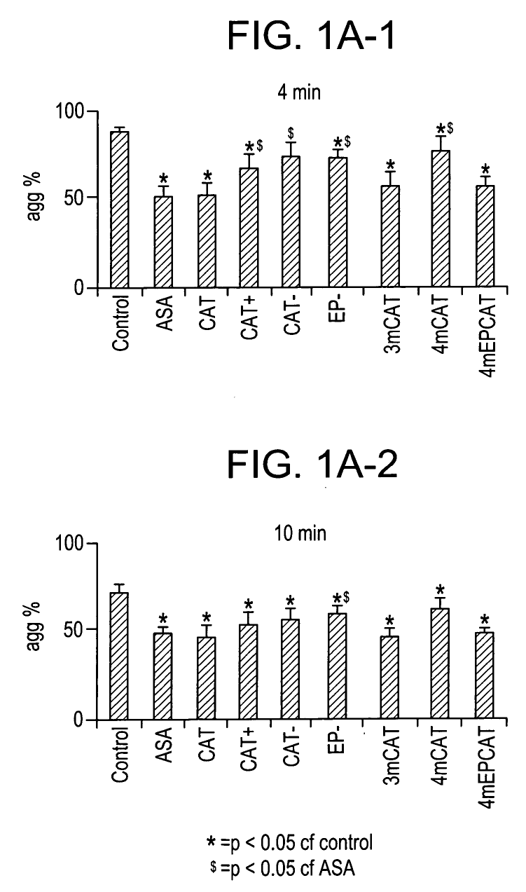

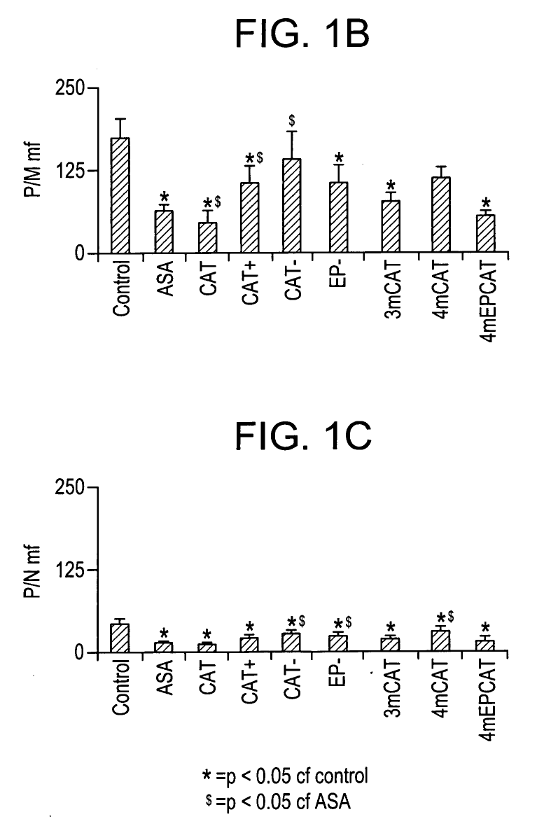

The absolute response of the blood from the different volunteers to collagen varied. This meant that the relative inhibitory effects of a flavanol was dependent on the volunteer's responsiveness to the particular collagen concentration used. For this reason it was decided that an appropriate means of analysing the results, for comparative purposes, would be to calculate the mean values for each flavanol irrespective of the collagen concentration used. The results are shown in FIG. 1. (Because, for each of the three blood samples three concentrations of collagen were used, the results are each the means (±sem) of nine individual values).

All of the flavanols inhibited collagen-induced platelet aggregation, with 3mCAT and 4mEPCAT showing significant effect. With regard to P/M most flavanols inhibited the conjugation again with 3mCAT and 4mEPCAT showing significant effect. For P/N, all flavanols significantly inhibited conjugate formation with 3mCAT and 4mEPCAT being among the most effective. ASA also effectively inhibited the aggregation, P/M and P/N.

From this point on it was decided to include measures of the extent of the activation of both platelets and leukocytes in the conjugates that formed following addition of collagen to blood. P-selectin (CD62P) was measured on the platelets associated with leukocytes in the conjugates that formed. Leukocyte activation was measured as the amount of CD11b that was expressed. Four-color analysis was used.

Comparison of the Effects of Flavanols on Collagen-Induced Aggregation, P/M, P/N, CD62P-M, CD62P-N, CD11b-M and CD11b-N

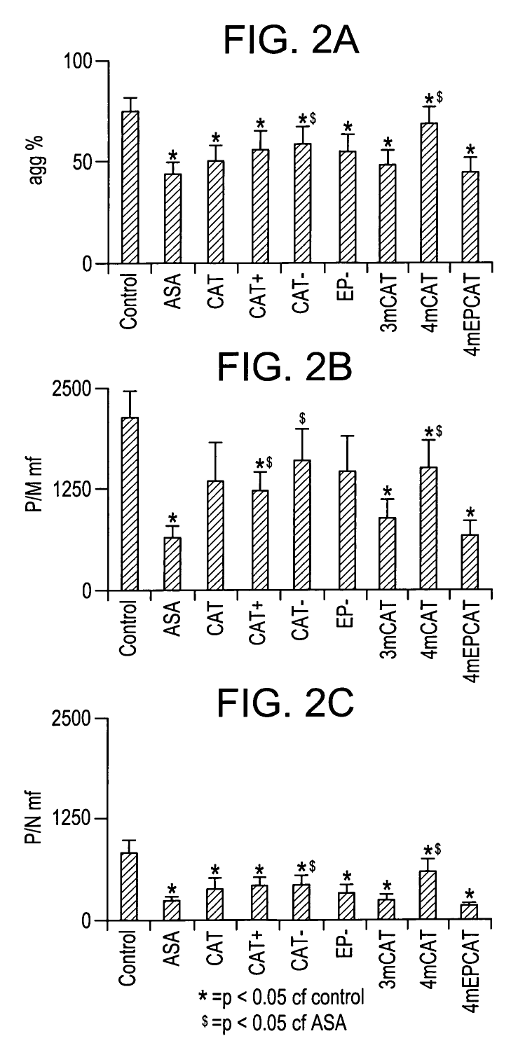

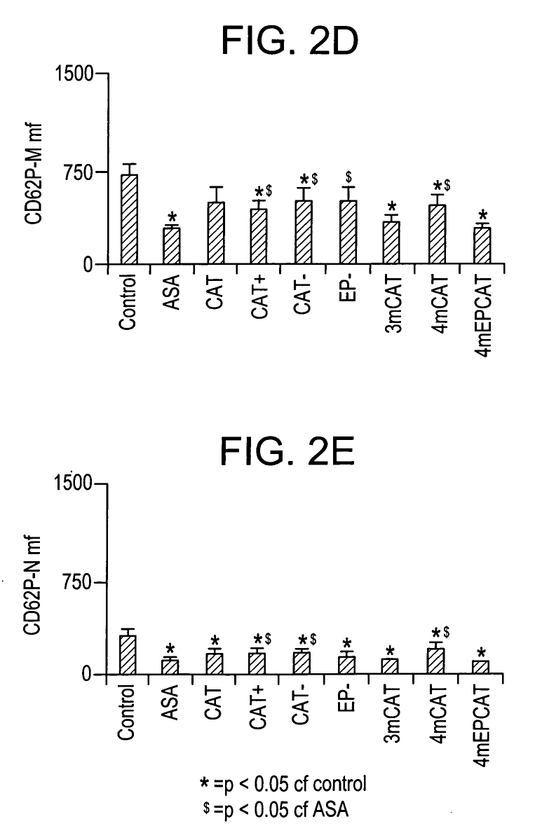

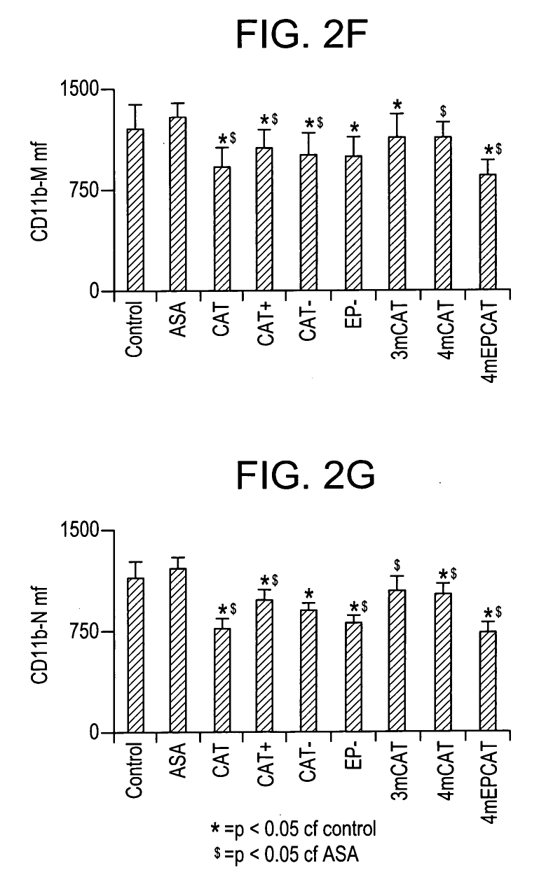

Blood was obtained from three different volunteers and the platelet aggregation (4 min) and platelet/leukocyte conjugate formation (10 min) was measured in response to collagen (0, 0.125, 0.25 and 0.5 μg/ml). At the same time the activation of platelets and leukocytes in the conjugates were measured by incubation with CD62P and CD11b antibodies. In these experiments aspirin was used at a concentration of 100 μM and derivatized flavanols and procyanidins at 0.3 mM with the exception of EP-(0.1 mM) and 4mCAT (0.05 mM). The results are shown in FIG. 2. As before, the analysis was performed by including all results for all three collagen concentrations and calculating the mean (±sem, n=9).

All flavanols inhibited collagen induced aggregation, again with 3mCAT and 4mEPCAT being among the most effective. Most flavanols inhibited P/M with 3mCAT and 4mEPCAT showing significant effect. P/N was inhibited by all flavanols also with 3mCAT and 4mEPCAT (used at 0.3 mM) being most effective. Platelet activation (CD62P) on monocytes and neutrophils was also best inhibited by 3mCAT and 4mEPCAT. The expected effects of aspirin were seen on all parameters.

Most flavanols inhibited leukocyte activation on both cell types (CD11b on monocytes and leukocytes), with 4mEPCAT being among the most effective. Aspirin also had no effect on CD11b.

### Example 3

### Effect of Derivatized Flavanols and Procyanidins on NO Production and Vasorelaxation

Alkylated compounds, obtained as described in Example 1, were investigated for their effect on nitric oxide (NO) production and vasorelaxation using serum-free human umbilical vein endothelial cell (HUVEC) culture system in vitro. NO production by endothelial cells and relaxation of pre-constricted aortic rings are two main markers for evaluating cardiovascular effects of test compounds.

In vitro Experiment

HUVECs obtained from a single donor were cultured in serum free, low protein (0.5 g/l), antibiotic-free cell culture medium supplemented with essential growth factors, nutrients and minerals. The cultured cell expressed endothelial markers (von Willebrand factor, CD31 antigen, uptake of Dil-Ac-LDL) and exhibited the typical “cobble-stone morphology” when grown to confluence. The cell culture medium was substituted with apo-transferrin, superoxide dismutase, and catalase to exclude secondary effects of test compounds involving their auto-oxidation mediated hydrogen peroxide formation.

Test compounds were evaluated with respect to their potential to acutely (2 hours) and chronically (5 doses given in a 24 h period) modulate NO production. Positive controls (acetylcholine and/or histamine) and negative control L-NNMA (NO synthase inhibitor) were included in all experiments. Cell counts and total protein were used to assess intra-assay variation. Potential toxic effects of tested compounds were also monitored (MTT reduction was measured).

NO production was evaluated by measuring the total amount of all major nitric oxide end products (NOx, including nitrate, nitrite, nitrosothiols) present in the cell culture medium. For this purpose NOx were directly reduced by vanadium (III) chloride/HCl at 95 degrees C. yielding NO. The amount of NO released from the culture medium was subsequently evaluated by measuring the chemiluminescence emitted during the stoichiometrical reaction between ozone and NO using NO Analyzer (Sievers Instruments, Inc. Boulder, Colo.).

The data presented herein were obtained from three experiments and were expressed as the concentration of NO present (in μmol/l) (as NOx) in the cell culture medium+/−standard deviation (SD). The data were corrected for the NOx intrinsically present in the fully supplemented cell culture medium and normalized with respect to the volume of media from which the sample was drawn. Data were analyzed using Student's t-test with a 95% level of confidence. P values equal to or less than 0.005 were defined as statistically significant.

For the acute effect test, HUVECs were incubated with a single dose of 4′-O-ethyl (−) epicatechin, 4′-O-methyl (−) catechin, 3′-O-methyl (−) catechin which may be prepared as described in Ex. 1 using (−) catechin as a starting material, for 2 and 24 hours at concentrations of 100 nM, 1 M, and 10 μM at 37 C and 5% CO₂. The alkylated compounds showed no statistically significant effect on NO production after 2 or 24 hours. Based on the MTT assay, the test compounds did not have toxic effects.

For the chronic effect test, HUVECs were incubated with 5 subsequent doses of test compounds, each for 24 hours. After each 24 hour treatment, culture medium was replaced. 4′-O-methyl (−) catechin, 3′-O-methyl (−) catechin showed no statistically significant effect on NO production. 4′-O-ethyl (−) epicatechin exhibited statistically significant increase in NO production (p=0.004) at 10 μM concentration.

Ex Vivo Experiment

Effect of 3′-O-methyl-(−)-catechin, 4′-O-methyl-(−)-catechin, 3′-O-ethyl-(−)-epicatechin and 4′-O-ethyl-(−)-epicatechin on endothelium-dependent relaxation is tested in an ex vivo experiment performed as previously described by Karim et al., J. Nutrl Suppl., 130 (8S): 2105S-2108S (2000), the relevant portions of which are hereby incorporated herein by reference. The advantage of using this method is that it assesses functional cardiovascular end points. The method is only able to assess acute events and does not allow for the identification of drug-induced protein expression/activity.

In summary, rabbit aortic rings are obtained from male New Zealand White rabbits. Following isolation, the rings are mounted in oxygenated Kreb's buffer, and are pre-constricted with NE (10⁻⁶ M). When the tension reaches a steady state, cumulative concentrations of the test compounds are applied (10-9 to 10-4 M).

A positive control acetylcholine (10⁻⁶M) and a negative control L-NAME are included in the experiment. Use of L-NAME, which is a NO synthase (NOS) inhibitor, allows for differentiating between endothelium dependent and endothelium independent relaxation events. Denuding of aortic rings represents a similar control. 400 U/mL of catalase is added into the aortic bath prior to the addition of each of the test compounds to ensure that the observed effects are not caused by hydrogen peroxide (H₂O₂) generation in the culture medium. The relaxation response is measured as a function of the decrease in the tension (g) exerted by the aortic rings over time. Data obtained are expressed as a percent relaxation of the norepinephrine (NE) constricted rings. The same statistical approach as described above is used. Dose response curves are obtained by plotting the average percent relaxation (+/−SE) against the concentrations used.

The results of the ex vivo screening showed no statistically significant effects of tested compounds.

What is claimed is:

1. A method of anti-platelet therapy or prophylaxis comprising administering to a subject in need thereof an effective amount of a derivatized flavanol having the following formula, or a pharmaceutically acceptable salt thereof, or a derivative thereof: 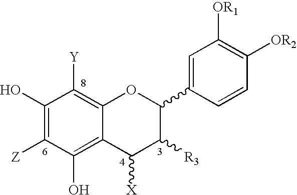
wherein
(i) R₁ or R₂ or both are selected from the group of: C₁ to C₄ alkyl, C₃ to C₄ alkenyl, and C₃ to C₄ alkynyl; with the proviso that when R₁ or R₂ or both are C₃ to C₄ alkenyl, or C₃ to C₄ alkynyl, the unsaturated carbons are separated by at least one carbon from the oxygen atom;
(ii) R₃ is -(α)-OH, -(β)-OH, -(α)-O-sugar, -(β)-O-sugar, -(α)-O-gallate, or -(β)-O-gallate;
(iii) each X, Y or Z is a hydrogen or a sugar; and
(iv) when R₁ or R₂ is not C₁ to C₄ alkyl, C₃ to C₄ alkenyl, or C₃ to C₄ alkynyl, it is a hydrogen; and
wherein the subject is a human or a veterinary animal.

2. The method of claim 1, wherein R₃ is -(α)-OH or -(β)-OH.

3. The method of claim 1, wherein R₃ is -(α)-O-gallate, or -(β)-O-gallate.

4. The method of claim 1, wherein of X, Y, and Z are hydrogen.

5. The method of claim 2, wherein of X, Y, and Z are hydrogen.

6. The method of claim 3, wherein of X, Y, and Z are hydrogen.

7. The method of claim 1, wherein R₁ or R₂ or both are methyl.

8. The method of claim 2, wherein R₁ or R₂ or both are methyl.

9. The method of claim 4, wherein R₁ or R₂ or both are methyl.

10. The method of claim 5, wherein R₁ or R₂ or both are methyl.

11. The method of claim 1, wherein the subject is human.

12. The method of claim 11, wherein R₃ is -(α)-OH or -(β)-OH.

13. The method of claim 11, wherein R₃ is -(α)-O-gallate, or -(β)-O-gallate.

14. The method of claim 11, wherein of X, Y, and Z are hydrogen.

15. The method of claim 12, wherein of X, Y, and Z are hydrogen.

16. The method of claim 13, wherein of X, Y, and Z are hydrogen.

17. The method of claim 11, wherein R₁ or R₂ or both are methyl.

18. The method of claim 12, wherein R₁ or R₂ or both are methyl.

19. The method of claim 14, wherein R₁ or R₂ or both are methyl.

20. The method of claim 15, wherein R₁ or R₂ or both are methyl.

21. The method of claim 11, wherein the human is suffering, or is at risk of suffering, from a condition selected from the group consisting of: thrombosis, plaque rupture, atherosclerosis, cardiovascular disease, coronary artery disease, myocardial ischemia, myocardial infarction, stable and unstable angina, acute occlusion, restenosis, vascular complications of diabetes, cognitive dysfunction or disorder, vascular circulation disorders, vascular circulation disorder of the brain, heart attack, cerebrovascular disease, stroke, initial transient ischemic attack, recurrent transient ischemic attack, ischemic complications, congestive heart failure, kidney failure, renal failure, peripheral artery disease, non-rheumatic atrial fibrillation and acute coronary syndrome.

22. The method of claim 11, wherein the human is suffering, or is at risk of suffering, from cardiovascular disease.

23. The method of claim 11, wherein the human is suffering, or is at risk of suffering, from vascular complications of diabetes.

24. The method of claim 11, wherein the human is suffering, or is at risk of suffering, from vascular circulation disorders.

25. The method of claim 11, wherein the human is suffering, or is at risk of suffering, from peripheral artery disease.

26. The method of claim 15, wherein the human is suffering, or is at risk of suffering, from a condition selected from the group consisting of: thrombosis, plaque rupture, atherosclerosis, cardiovascular disease, coronary artery disease, myocardial ischemia, myocardial infarction, stable and unstable angina, acute occlusion, restenosis, vascular complications of diabetes, cognitive dysfunction or disorder, vascular circulation disorders, vascular circulation disorder of the brain, heart attack, cerebrovascular disease, stroke, initial transient ischemic attack, recurrent transient ischemic attack, ischemic complications, congestive heart failure, kidney failure, renal failure, peripheral artery disease, non-rheumatic atrial fibrillation and acute coronary syndrome.

27. The method of claim 20, wherein the human is suffering, or is at risk of suffering, from a condition selected from the group consisting of: thrombosis, plaque rupture, atherosclerosis, cardiovascular disease, coronary artery disease, myocardial ischemia, myocardial infarction, stable and unstable angina, acute occlusion, restenosis, vascular complications of diabetes, cognitive dysfunction or disorder, vascular circulation disorders, vascular circulation disorder of the brain, heart attack, cerebrovascular disease, stroke, initial transient ischemic attack, recurrent transient ischemic attack, ischemic complications, congestive heart failure, kidney failure, renal failure, peripheral artery disease, non-rheumatic atrial fibrillation and acute coronary syndrome.

28. The method of claim 1, wherein the derivatized flavanol is selected form the group consisting of 3′-O-methyl-(+)catechin, 3′-O-methyl-(−)-epicatechin, 4′-O-methyl-(+)-catechin, 4′-O-methyl-(−)-epicatechin, 3′-O—, 4′-O-dimethyl-(+)-catechin, and 3′-O—, 4′-O-dimethyl-(−)-epicatechin.

29. The method of claim 28, wherein the subject is human.

30. The method of claim 29, wherein the human is suffering, or is at risk of suffering, from a condition selected from the group consisting of: thrombosis, plaque rupture, atherosclerosis, cardiovascular disease, coronary artery disease, myocardial ischemia, myocardial infarction, stable and unstable angina, acute occlusion, restenosis, vascular complications of diabetes, cognitive dysfunction or disorder, vascular circulation disorders, vascular circulation disorder of the brain, heart attack, cerebrovascular disease, stroke, initial transient ischemic attack, recurrent transient ischemic attack, ischemic complications, congestive heart failure, kidney failure, renal failure, peripheral artery disease, non-rheumatic atrial fibrillation and acute coronary syndrome.
